(19) United States
(12) Patent Application Publication
(10) Pub. No.: US 2016/0157831 A1
(43) Pub. Date: Jun. 9, 2016

(54) APPARATUS AND METHOD FOR SUPPORTING COMPUTER-AIDED DIAGNOSIS

(71) Applicant: SAMSUNG ELECTRONICS CO., LTD., Suwon-si (KR)

(72) Inventors: Byung Kon KANG, Gwangju-si (KR); Hyoung Min PARK, Seoul (KR)

(73) Assignee: SAMSUNG ELECTRONICS CO., LTD., Suwon-si (KR)

(21) Appl. No.: 14/958,312

(22) Filed: Dec. 3, 2015

(30) Foreign Application Priority Data
Dec. 3, 2014 (KR) 10-2014-0172348

(51) Int. Cl. A61B 8/08; A61B 8/14; A61B 8/00

(52) U.S. Cl. CPC A61B 8/5215; A61B 8/46; A61B 8/14

(57) ABSTRACT

An apparatus and a method for supporting Computer-Aided Diagnosis (CAD) are provided. The apparatus includes a cause analyzer configured to analyze a cause of a reliability of a diagnosis result of a medical image being lower than a level, the medical image being captured using a probe, and a guide information generator configured to generate guide information of an operation of the probe based on the analyzed cause.

### CROSS-REFERENCE TO RELATED APPLICATION

This application claims priority from Korean Patent Application No. 10-2014-0172348, filed on Dec. 3, 2014, in the Korean Intellectual Property Office, the disclosure of which is incorporated herein by reference in its entirety.

### BACKGROUND

1. Field

Apparatuses and methods consistent with exemplary embodiments relate to an apparatus and a method for supporting Computer-Aided Diagnosis (CAD).

2. Description of the Related Art

Computer-Aided Diagnosis (CAD) systems are widely used for analysis of medical images. Once a doctor presses a probe against a diseased body portion, the real-time CAD system acquires ultrasound images in real time, and if a lesion or a region suspected as having a disease is detected from the acquired images, the CAD system informs the doctor of a diagnosis result regarding a lesion or a suspected region.

However, in the real-time CAD, diagnosis performed by moving a probe is significantly affected by time. That is, in the real-time CAD, diagnosis results are calculated within a short period of time during which several frames are acquired by moving a probe, thereby reducing diagnosis accuracy. In this case, a user that performs diagnosis analyzes an image and determines whether the image has an unclear region, and requests the CAD system to analyze the unclear region more deeply, in which in response to the request, the CAD system stops real-time analysis, and performs analysis in non-real time.

### SUMMARY

Exemplary embodiments may address at least the above problems and/or disadvantages and other disadvantages not described above. Also, the exemplary embodiments are not required to overcome the disadvantages described above, and an exemplary embodiment may not overcome any of the problems described above.

According to an aspect of an exemplary embodiment, there is provided an apparatus for supporting computer-aided diagnosis, the apparatus including a cause analyzer configured to analyze a cause of a reliability of a diagnosis result of a medical image being lower than a level, the medical image being captured using a probe, and a guide information generator configured to generate guide information of an operation of the probe based on the analyzed cause.

The reliability of the diagnosis result may include at least one among a detection reliability and a determination reliability, the detection reliability indicating whether lesions are detected without omission, and the determination reliability indicating an accuracy of determinations whether the detected lesions are malignant or benign.

The detection reliability may be in inverse proportion to a moving speed of the probe.

The cause analyzer may be configured to analyze the cause based on at least one among a moving speed of the probe and a quality of the medical image.

In response to the cause analyzer analyzing the cause to be a moving speed of the probe being above a level, the guide information generator may be configured to generate the guide information of the moving speed of the probe.

In response to the cause analyzer analyzing the cause to be a quality of the medical image, the guide information generator may be configured to generate the guide information of at least one among an angle and a pressure of the probe.

The apparatus may further include an output interface configured to output the generated guide information, using at least one among an acoustic method, a visual method, and a tactile method.

The output interface may be configured to output the generated guide information as at least one among an image and a text of the operation of the probe.

According to an aspect of another exemplary embodiment, there is provided a method of supporting computer-aided diagnosis, the method including analyzing a cause of a reliability of a diagnosis result of a medical image being lower than a level, the medical image being captured using a probe, and generating guide information of an operation of the probe based on the analyzed cause.

The analyzing may include analyzing the cause based on at least one among a moving speed of the probe and a quality of the medical image.

In response to the analyzing the cause to be a moving speed of the probe being above a level, the generating may include generating the guide information of the moving speed of the probe.

In response to the analyzing the cause to be a quality of the medical image, the generating may include generating the guide information of at least one among an angle and a pressure of the probe.

The method may further include outputting the generated guide information, using at least one among an acoustic method, a visual method, and a tactile method.

The outputting may include outputting the generated guide information as at least one among an image and a text of the operation of the probe.

A non-transitory computer-readable storage medium may store a program including instructions for causing a computer to perform the method.

According to an aspect of another exemplary embodiment, there is provided a computer-aided diagnosis apparatus including an image acquirer configured to acquire a medical image, using a probe, a lesion determiner configured to determine a lesion based on the acquired medical image, and a diagnosis supporting portion configured to analyze a cause of a reliability of the determined lesion being less than a level, to be at least one among a moving speed, an angle, and a pressure of the probe, and generate guide information of at least one among the moving speed, the angle, and the pressure of the probe based on the analyzed cause.

The diagnosis supporting portion may be further configured to output the generated guide information, using at least one among an acoustic method, a visual method, and a tactile method.

The diagnosis supporting portion may be configured to analyze the cause to be the moving speed of the probe in response to the moving speed being greater than a level, analyze the cause to be the angle of the probe in response to the angle being different than a right angle to the lesion, and analyze the cause to be the pressure of the probe in response to the pressure being less than a level.

### BRIEF DESCRIPTION OF THE DRAWINGS

The above and/or other aspects will become more apparent by describing exemplary embodiments with reference to the accompanying drawings, in which:

FIG. 1 is a block diagram illustrating an apparatus for supporting Computer-Aided Diagnosis (CAD), according to an exemplary embodiment;

FIG. 2 is a diagram illustrating a moving speed of a probe causing reliability degradation;

FIG. 3 is a diagram illustrating an angle of a probe causing reliability degradation;

FIG. 4 is a diagram illustrating a pressure of a probe causing reliability degradation;

FIG. 5 is a block diagram illustrating an apparatus for supporting CAD, according to another exemplary embodiment;

FIGS. 6A, 6B, and 6C are diagrams illustrating methods of visually outputting guide information, according to exemplary embodiments;

FIG. 7 is a flowchart illustrating a method of supporting CAD, according to an exemplary embodiment;

FIG. 8 is a block diagram illustrating a CAD apparatus, according to an exemplary embodiment.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments may be practiced without those specifically defined matters. Also, well-known functions or constructions may not be described in detail because they would obscure the description with unnecessary detail.

Hereinafter, exemplary embodiments of an apparatus and a method for supporting Computer-Aided Diagnosis (CAD) will be described with reference to the accompanying drawings.

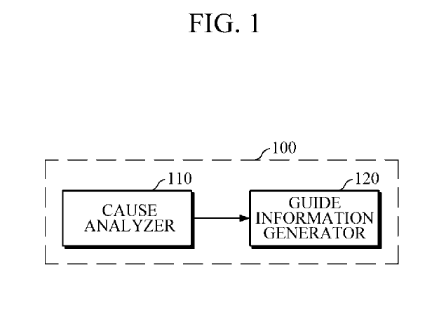

FIG. 1 is a block diagram illustrating an apparatus 100 for supporting CAD, according to an exemplary embodiment.

Referring to FIG. 1, the apparatus 100 for supporting CAD includes a cause analyzer 110 and a guide information generator 120.

The cause analyzer 110 analyzes a cause of low reliability in response to a reliability of a diagnosis result being lower than a predetermined level.

A reliability of a diagnosis result may include at least one of a detection reliability and a determination reliability, in which the detection reliability indicates whether lesions are detected without omission, and the determination reliability indicates an accuracy of determinations whether detected lesions are malignant or benign. In this case, the detection reliability may be in inverse proportion to a moving speed of a probe.

Causes of low reliability may include characteristics of a lesion, performance of a probe, methods of operating a probe, incompleteness of an algorithm for analysis, and the like, among which characteristics of a lesion, performance of a probe, and incompleteness of an algorithm are intrinsic characteristics of a diagnosis subject and a diagnosis device, and thus, do not change a reliability in real time. However, methods of operating a probe may be changed in real time by a user, such that when an operation method is changed while real-time diagnosis is performed, a reliability may be changed accordingly. Thus, operation methods may be a cause that may affect reliabilities of diagnosis results.

In an exemplary embodiment, the cause analyzer 110 may analyze a cause of low reliability based on a quality of medical images. The quality of medical images may be determined depending on a degree of noise included in an image signal or on a degree of distortion of medical images. For example, in the case where a noise that is above a predetermined level is included in a signal, or where an image distortion occurs due to absorption, reflection, scattering, and the like, of signals, reliabilities of diagnosis results on medical images may be reduced. The quality of medical images may be affected by an angle of a probe at a time of measurement, by a pressure of a probe at the time of measurement, and the like.

Accordingly, once the quality of medical images is determined to be low upon analyzing the quality, the cause analyzer 110 may determine that a low quality of medical images may be a cause of low reliability.

The case where a pressure or an angle of a probe is a cause of low reliability will be described below with the following examples.

For example, an angle of a probe at the time of measurement may reduce reliabilities of diagnosis results. In the case where measurement is performed by using a probe at an angle, a noise level may be increased. The probe receives a signal that is reflected from body tissues. Where measurement is performed with a probe and a body tissue aligning at an inappropriate angle, strength of a signal reflected into the probe may be reduced. That is, when an incident angle of a signal is right-angled, the highest signal strength may be obtained. However, if an incident angle gets smaller, a signal is reflected in an opposite direction of a probe without reaching the probe. In this case, a signal strength gets smaller, and a noise may result in quality degradation of medical images. Accordingly, an angle of a probe at the time of measurement may cause low reliability.

In another example, reliabilities of diagnosis results may be reduced due to a pressure of a probe at the time of measurement. In a case where a probe is not pressed with (i.e., less than) a sufficient pressure, a noise component may be increased in a signal received by using the probe, and as a distance between the probe and a lesion gets larger, the signal may be attenuated or distorted. That is, a pressure of a probe at the time of measurement may increase attenuation, distortion, and noise of a signal, thereby degrading quality of medical images. Accordingly, a pressure of a probe at the time of measurement may be a cause of low reliability.

In another exemplary embodiment, the cause analyzer 110 may analyze a cause of low reliability based on a moving speed of a probe. For example, in the case where a moving speed of a probe is determined to be above a predetermined level, the cause analyzer 110 may analyze that a fast moving speed of a probe is the cause of low reliability.

The case where a moving speed of a probe is a cause of low reliability will be described below with the following examples.

For example, in the case of a fast moving speed of a probe, a distance between frames acquired by a probe may get larger. In this case, a small lesion may be positioned between image frames acquired by a probe, and a medical image of the small lesion may not be acquired, thereby omitting detection of the lesion. Accordingly, in the case of a fast moving speed of a probe, the moving speed may be a cause of low reliabilities of diagnosis results, i.e., low detection reliability.

In another example, in the case of a fast moving speed of a probe, a reliability may be lowered due to a relationship between a frame processing speed and a number of frames per second of an apparatus for supporting CAD. In the case where a frame processing speed of a diagnosis algorithm is slower than the number of frames per second of the apparatus for supporting CAD, the diagnosis algorithm may omit a medical image frame. In this case, the diagnosis algorithm may not process an image frame of a small lesion, and may omit detection of the small lesion. Accordingly, a fast moving speed of a probe may be a cause of low reliabilities of diagnosis results, i.e., low detection reliability.

The cause analyzer 110 may detect a moving speed of a probe.

In an exemplary embodiment, the cause analyzer 110 may detect a speed of a probe by using medical images to analyze a cause of low reliability based on a moving speed of a probe. For example, the cause analyzer 110 may detect a speed of a probe based on a difference between a sum of image intensities for pixels of a previous image frame and a sum of image intensities for pixels of a current image frame acquired through a probe. In another example, the cause analyzer 110 may detect a speed of a probe based on a difference between histograms of a previous image frame and a current image frame. In still another example, the cause analyzer 110 may detect a speed of a probe based on a change in information, such as salient regions of a previous image frame, a current image frame, and the like. In yet another example, the cause analyzer 110 may estimate a speed of a probe indirectly from a resolution of a current frame image.

In another exemplary embodiment, the cause analyzer 110 may detect a moving speed of a probe by using an accelerometer sensor and/or the like mounted in the probe.

The guide information generator 120 generates guide information associated with operations of a probe based on the analyzed cause of low reliability.

In an exemplary embodiment, in the case where an analyzed cause of low reliability is a moving speed of a probe, the guide information generator 120 may generate guide information associated with a moving speed of a probe.

For example, in response to receiving, from the cause analyzer 110, information indicating that a moving speed of a probe is fast, the guide information generator 120 may generate guide information for a user to reduce a moving speed of a probe. Further, in the case where diagnosis may not be performed appropriately due to a fast moving speed of a probe, the guide information generator 120 may generate guide information for a user to analyze an undiagnosed portion by using a probe.

In another exemplary embodiment, in the case where an analyzed cause of low reliability is a low quality of medical images caused by an inappropriate angle or pressure of a probe, the guide information generator 120 may generate guide information associated with at least one of an angle and a pressure of a probe.

For example, in response to receiving, from the cause analyzer 110, information indicating that a medical image has a low quality due to an inappropriate measurement angle of a probe, the guide information generator 120 may generate guide information for a user to change a measurement angle of a probe. Further, in the case where an appropriate diagnosis is not performed due to a measurement angle of a probe, the guide information generator 120 may generate guide information for a user to diagnose an undiagnosed portion using a probe at a changed angle.

In another example, in response to receiving, from the cause analyzer 110, information indicating that an inappropriate measurement pressure of probe causes a low quality of medical images, the guide information generator 120 may provide a user with a signal to change a measurement pressure of a probe. Further, in the case where an appropriate diagnosis is not performed due to a measurement angle of a probe, the guide information generator 120 may provide a user with a signal to diagnose an undiagnosed portion using a probe at a changed pressure.

In still another exemplary embodiment, in response to receiving, from the cause analyzer 110, information that includes one or more causes, the guide information generator 120 may generate guide information for operation of a probe based on one cause that is considered to most affect a reliability, and may generate one or more types of guide information by combining the analyzed causes.

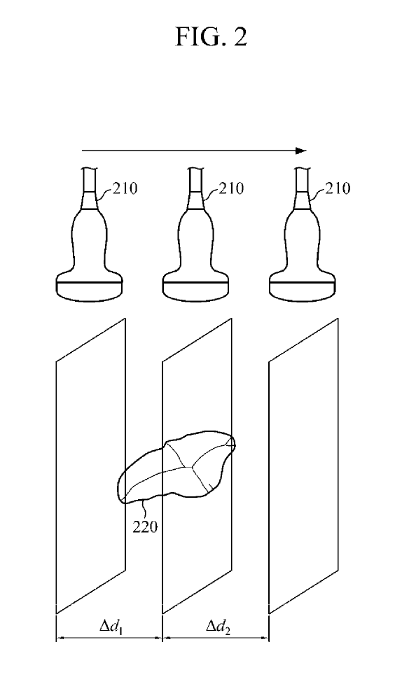

FIG. 2 is a diagram illustrating a moving speed of a probe 210 causing reliability degradation.

Referring to FIG. 2, in the case of a fast moving speed of the probe 210, reliabilities of diagnosis results may be lowered. That is, in the case where a lesion 220 is small, the lesion 220 may be located between a distance (Δd₁) of images acquired by the probe 210. In this case, a medical image of the lesion 220 may not be acquired, and thus the lesion 220 may not be detected or recognized.

For example, in the case where a frame processing speed is 10 frames per second (fps) and a moving speed of the probe 210 is 2 cm per second, Δd₁ is 2 mm. In this case, a lesion having a size smaller than 2 mm may be omitted.

Accordingly, a moving speed of the probe 210 may be a cause of low reliability, because a fast moving speed of the probe 210 may result in lowered reliabilities of diagnosis results, i.e., lowered detection reliability.

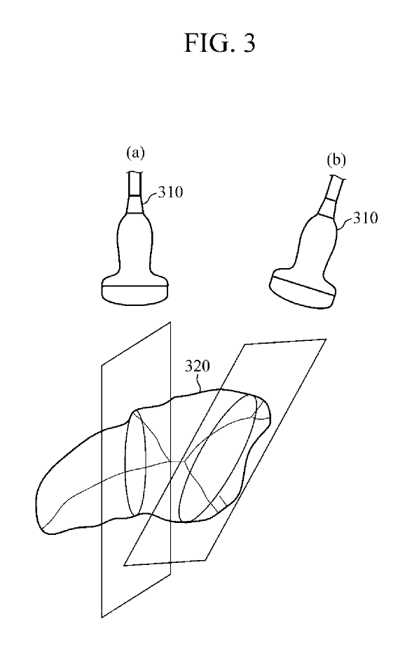

FIG. 3 is a diagram illustrating an angle of a probe 310 causing reliability degradation.

Referring to FIG. 3, in the case where a measurement angle of the probe 310 is not accurate, reliabilities of diagnosis results may be lowered. For example, in the case where an angle of the probe 310 is changed, a quality of medical images may be degraded. In this case, diagnosis results obtained by using a diagnosis algorithm for diagnosing a lesion 320 may not be appropriate to determine whether the lesion 320 is malignant or benign. For example, if the lesion 320 is measured at an oblique angle to the lesion 320 as illustrated in portion (b) of FIG. 3, an incident angle is reduced, and a signal is reflected in an opposite direction of the probe 310, such that the signal may not be received by the probe 310. In this case, a signal strength is reduced, and a noise may cause low quality of medical images. As a result, diagnosis results obtained by using a diagnosis algorithm may not be appropriate to determine whether the lesion 320 is malignant or benign. In this case, by measuring the lesion 320 at a right angle to the lesion 320 as illustrated in portion (a) of FIG. 3, the highest signal strength may be obtained, so that reliabilities of diagnosis results may be improved.

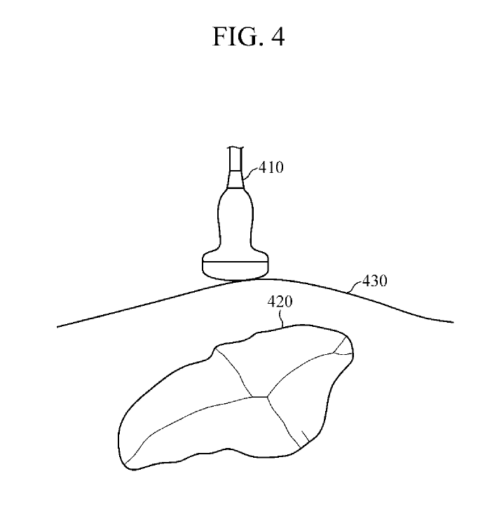

FIG. 4 is a diagram illustrating a pressure of a probe 410 causing reliability degradation.

Referring to FIG. 4, in the case where a measurement pressure of the probe 410 is not accurate, reliabilities of diagnosis results may be lowered. For example, in the case where the probe 410 is not accurately pressed against a portion 430 to be measured, noise components may be increased in a signal received by using the probe 410.

Further, distortion and noise may be generated due to a distance between the probe 410 and a lesion 420. That is, in the case where a distance between the probe 410 and the lesion 420 is small, signal strength received by the probe 410 is increased, with fewer factors that may disrupt a signal between the probe 410 and the lesion 420. In this case, distortion and noise may be reduced, thereby increasing a quality of medical images, and leading to increased reliabilities of diagnosis results. By contrast, if a sufficient pressure is not applied to the probe 410, a distance between the probe 410 and the lesion 420 gets larger, such that noise and distortion may be caused. As a result, the quality of medical images may be reduced, thereby reducing reliabilities of diagnosis results.

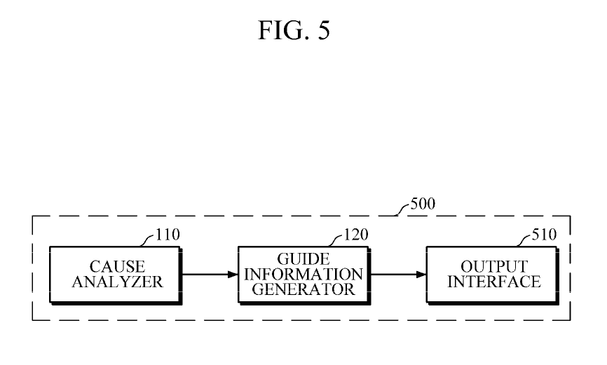

FIG. 5 is a block diagram illustrating an apparatus 500 for supporting CAD, according to another exemplary embodiment.

Referring to FIG. 5, the apparatus 500 for supporting CAD further includes an output interface 510 in addition to the cause analyzer 110 and the guide information generator 120 of FIG. 1.

In the case where reliabilities of diagnosis results is below a predetermined level, the output interface 510 notifies a user by using an acoustic method, a visual method, a tactile method, and/or the like. The output interface 510 outputs guide information generated by the guide information generator 120.

In an exemplary embodiment, the output interface 510 may output the generated guide information by using at least one of the acoustic method, the visual method, and the tactile method. For example, the output interface 510 may output guide information by using a voice, an image, vibration, texts, video, and the like.

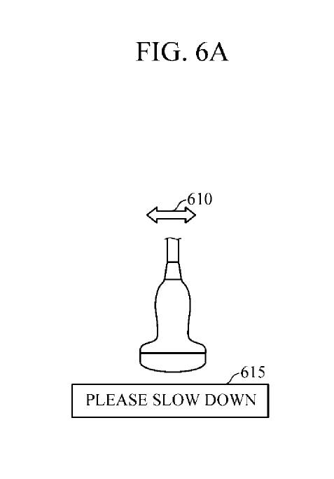

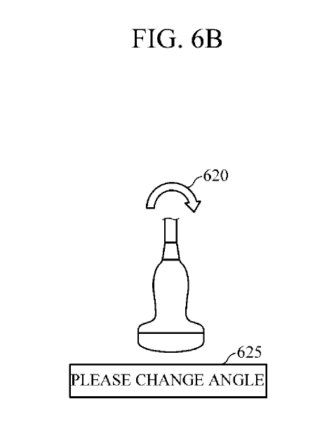

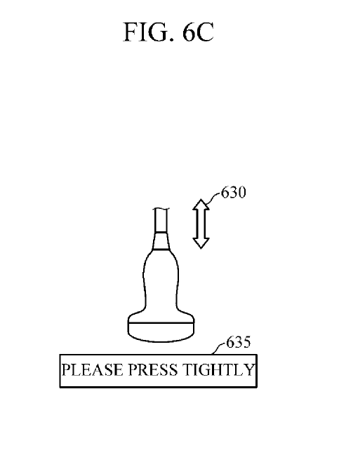

FIGS. 6A, 6B, and 6C are diagrams illustrating methods of visually outputting guide information, according to exemplary embodiments. FIG. 6A illustrates an example of outputting guide information associated with a speed of a probe, FIG. 6B illustrates an example of outputting guide information associated with an angle of a probe, and FIG. 6C illustrates an example of outputting guide information associated with a pressure of a probe.

Referring to FIG. 6A, in the case where it is determined that a speed of a probe is the cause of low reliability, and the guide information generator 120 generates guide information associated with a speed of a probe, the output interface 510 may output on a screen an image 610 to slow down a speed of a probe. Further, the output interface 510 may output on a screen a text 615 to slow down a speed of a probe.

The example illustrated in FIG. 6A is an exemplary embodiment, and the present disclosure is not limited thereto. That is, the output interface 510 may output a video that shows a probe moving from left to right on a screen as a sign to slow down a probe. The output interface 510 may output speech, such as “please slow down a probe,” through a speaker, and may generate vibration in a probe.

Referring to FIG. 6B, in the case where it is determined that a measurement angle of a probe is the cause of low reliability, and the guide information generator 120 generates guide information associated with a measurement angle of a probe, the output interface 510 may output on a screen an image 620 to change a measurement angle of a probe. Further, the output interface 510 may generate on a screen a text 625 to change a measurement angle of a probe.

The example illustrated in FIG. 6B is an exemplary embodiment, and the present disclosure is not limited thereto. That is, the output interface 510 may output a video showing an image of a probe that moves from left to right while changing an angle. The output interface 510 may output speech, such as “please change a measurement angle of a probe,” through a speaker, and may generate vibration in a probe.

Referring to FIG. 6C, in the case where it is determined that a pressure of a probe is the cause of low reliability, and the guide information generator 120 generates guide information associated with a pressure of a probe, the output interface 510 may output on a screen an image 630 to change a measurement pressure of a probe. Further, the output interface 510 may output on a screen a text 635 to change a measurement pressure of a probe.

The example illustrated in FIG. 6C is an exemplary embodiment, and the present disclosure is not limited thereto. That is, the output interface 510 may output a video showing an image of a probe that moves up and down as a sign to change a pressure of a probe. Further, the output interface 510 may output speech, such as “please further attach the probe,” through a speaker, and may generate vibration in a probe.

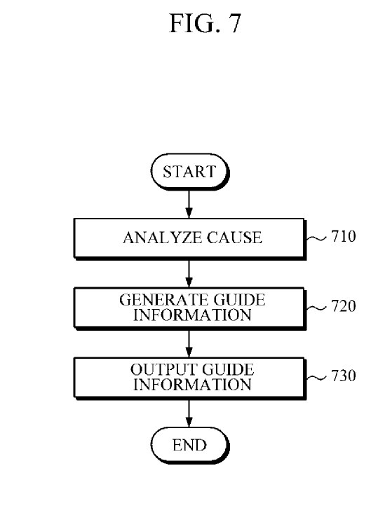

FIG. 7 is a flowchart illustrating a method of supporting CAD, according to an exemplary embodiment.

Referring to FIG. 7, in operation 710, the method of supporting CAD according to an exemplary embodiment includes analyzing a cause of low reliability in response to reliabilities of diagnosis results being below a predetermined level. For example, the apparatuses 100 and 500 of supporting CAD may analyze causes of low reliability based on a moving speed of a probe, a quality of medical images, and the like.

A reliability of a diagnosis result may include at least one of a detection reliability and a determination reliability, in which the detection reliability indicates whether lesions are detected without omission, and the determination reliability indicates an accuracy of determinations whether detected lesions are malignant or benign. The detection reliability may be in inverse proportion to a moving speed of a probe.

In operation 720, based on the analyzed cause of low reliability, the method includes generating guide information associated with operations of a probe.

In an exemplary embodiment, upon determination that a moving speed of a probe is the cause of low reliability, the apparatuses 100 and 500 for supporting CAD may generate guide information associated a moving speed of a probe.

For example, upon determination that a moving speed of a probe is fast, the apparatuses 100 and 500 for supporting CAD may generate guide information to slow down a probe. Further, in the case where diagnosis may not be performed appropriately due to a fast moving speed of a probe, the apparatuses 100 and 500 for supporting CAD may generate guide information for a user to analyze an undiagnosed portion by using a probe.

In another exemplary embodiment, upon determination that the quality of medical images is the cause of low reliability, the apparatuses 100 and 500 for supporting CAD may generate guide information associated with at least one of an angle and a pressure of a probe.

For example, upon determination that the quality of medical images is reduced due to an inappropriate angle of a probe, the apparatuses 100 and 500 for supporting CAD may generate guide information to change a measurement angle of a probe. Further, in the case where diagnosis may not be performed appropriately due to a measurement angle of a probe, the apparatuses 100 and 500 for supporting CAD may generate guide information for a user to analyze an undiagnosed portion at a changed angle of a probe.

In another example, upon determination that the quality of medical images is reduced due to an inappropriate pressure of a probe, the apparatuses 100 and 500 for supporting CAD may generate guide information for a user to change a measurement pressure of a probe. Further, in the case where diagnosis may not be performed appropriately due to a measurement pressure of a probe, the apparatuses 100 and 500 for supporting CAD may generate guide information for a user to analyze an undiagnosed portion at a changed pressure of a probe.

In still another exemplary embodiment, if there are one or more causes analyzed in the analysis of causes of low reliability, the apparatuses 100 and 500 for supporting CAD may generate guide information associated with operations of a probe based on one cause that most affects a reliability. Further, the apparatuses 100 and 500 for supporting CAD may generate one or more types of guide information by combining one or more of the analyzed causes.

In operation 730, the method includes outputting the generated guide information. For example, the apparatuses 100 and 500 for supporting CAD may output the generated guide information by using at least one of an acoustic method, a visual method, and a tactile method.

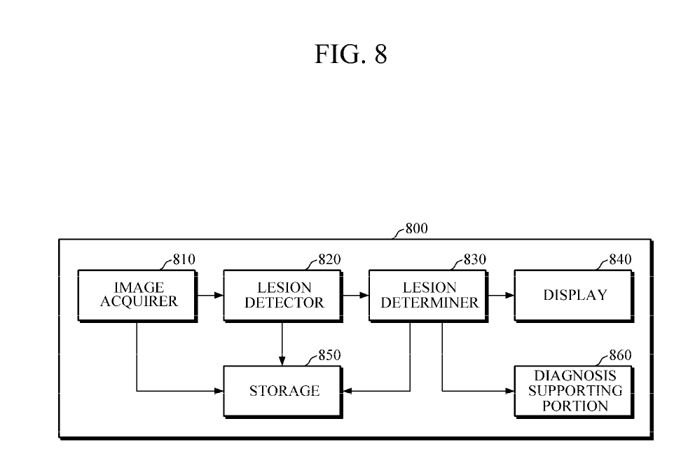

FIG. 8 is a block diagram illustrating a CAD apparatus, according to an exemplary embodiment.

Referring to FIG. 8, the CAD apparatus 800 includes an image acquirer 810, a lesion detector 820, a lesion determiner 830, a display 840, a storage 850, and a diagnosis supporting portion 860.

The image acquirer 810 acquires medical images of a patient's diseased area by using a probe. The medical images may be ultrasound images sequentially acquired in real time in units of frames.

The lesion detector 820 detects a location and an approximate size of a lesion from medical images of a patient. For example, the lesion detector 820 may detect a lesion by using various detection methods, such as an automatic detection algorithm, a detection method by receiving a user's input, and/or the like.

The lesion detection algorithm may include AdaBoost, Deformable Part Models (DPM), Deep Neural Network (DNN), Convolutional Neural Network (CNN), Overfeat, Sparse Coding, and the like, which is an example, and is not limited thereto.

The lesion determiner 830 extracts characteristic values of a lesion from a lesion area detected by the lesion detector 820. For example, the characteristic values of a lesion may be lesion characteristics primarily extracted from a detected lesion area, such as a shape, a margin, an ultrasound echo pattern, and the like, and may be values that may be obtained by calculating based on the primarily extracted lesion characteristics. Further, the lesion determiner 830 determines a lesion by using extracted lesion characteristic values and a diagnosis model stored in the storage 850.

The diagnosis model may be generated by machine learning using characteristic values extracted from a plurality of diagnosis images collected in advance, and the generated diagnosis model may be stored in a database inside or outside the lesion determiner 830.

The machine learning algorithm may include artificial neural network, decision tree, genetic algorithm (GA), genetic programming (GP), Gaussian process regression, linear discriminant analysis, K-Nearest Neighbor (K-NN), Perceptron, a Radial Basis Function Network, Support Vector Machine (SVM), deep-learning, and the like, which are illustrative examples, and the machine learning algorithm is not limited thereto.

The display 840 outputs, on a screen, medical images and determination results of lesions.

Further, the display 840 outputs detection results of lesions on a screen. When displaying detection results of lesions, the display 840 displays a detected lesion in a bounding box, and may display a location of a lesion by marking a lesion with a cross at a center thereof. However, the displaying of a lesion is not limited thereto, and a lesion may be displayed with distinguishable markers of various shapes, such as a circle, a triangle, and/or the like, and may be coded with various colors.

Further, the display 840 outputs, on a screen, guide information of the output interface 510 of FIG. 5.

The storage 850 may store medical images, detection results of lesions, determination results of lesions, image analysis algorithms, a diagnosis model, and the like.

The storage 850 may include flash memory type, hard disk type, multi-media card micro type, card type memory (e.g., SD or XD memory, etc.), random access memory (RAM), static random access memory (SRAM), read-only memory (ROM), electrically erasable programmable read only memory (EEPROM), programmable read only memory (PROM), magnetic memory, magnetic disks, optical discs, and/or the like.

The diagnosis supporting portion 860 may be an example of the apparatuses 100 and 500 for supporting CAD in FIGS. 1 and 5, respectively. That is, the diagnosis supporting portion 860 analyzes causes of low reliability when reliabilities of diagnosis results on medical images is below a predetermined level, and based on the analyzed causes, the diagnosis supporting portion 860 generates guide information associated with operations of a probe.

While not restricted thereto, an exemplary embodiment can be embodied as computer-readable code on a computer-readable recording medium. For example, a control program that controls the above-described operations of the apparatuses 100 or 500 may be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an exemplary embodiment may be written as a computer program transmitted over a computer-readable transmission medium, such as a carrier wave, and received and implemented in general-use or special-purpose digital computers that execute the programs. Moreover, it is understood that in exemplary embodiments, one or more units or portions of the above-described apparatuses 100 and 500 can include circuitry, a processor, a microprocessor, etc., and may execute a computer program stored in a computer-readable medium.

The foregoing exemplary embodiments and advantages are examples and are not to be construed as limiting. The present teaching may be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for supporting computer-aided diagnosis, the apparatus comprising:
a cause analyzer configured to analyze a cause of a reliability of a diagnosis result of a medical image being lower than a level, the medical image being captured using a probe; and
a guide information generator configured to generate guide information of an operation of the probe based on the analyzed cause.

2. The apparatus of claim 1, wherein the reliability of the diagnosis result comprises at least one among a detection reliability and a determination reliability, the detection reliability indicating whether lesions are detected without omission, and the determination reliability indicating an accuracy of determinations whether the detected lesions are malignant or benign.

3. The apparatus of claim 2, wherein the detection reliability is in inverse proportion to a moving speed of the probe.

4. The apparatus of claim 1, wherein the cause analyzer is configured to analyze the cause based on at least one among a moving speed of the probe and a quality of the medical image.

5. The apparatus of claim 1, wherein in response to the cause analyzer analyzing the cause to be a moving speed of the probe being above a level, the guide information generator is configured to generate the guide information of the moving speed of the probe.

6. The apparatus of claim 1, wherein in response to the cause analyzer analyzing the cause to be a quality of the medical image, the guide information generator is configured to generate the guide information of at least one among an angle and a pressure of the probe.

7. The apparatus of claim 1, further comprising an output interface configured to output the generated guide information, using at least one among an acoustic method, a visual method, and a tactile method.

8. The apparatus of claim 7, wherein the output interface is configured to output the generated guide information as at least one among an image and a text of the operation of the probe.

9. A method of supporting computer-aided diagnosis, the method comprising:
analyzing a cause of a reliability of a diagnosis result of a medical image being lower than a level, the medical image being captured using a probe; and
generating guide information of an operation of the probe based on the analyzed cause.

10. The method of claim 9, wherein the reliability of the diagnosis result comprises at least one among a detection reliability and a determination reliability, the detection reliability indicating whether lesions are detected without omission, and the determination reliability indicating an accuracy of determinations whether the detected lesions are malignant or benign.

11. The method of claim 10, wherein the detection reliability is in inverse proportion to a moving speed of the probe.

12. The method of claim 9, wherein the analyzing comprises analyzing the cause based on at least one among a moving speed of the probe and a quality of the medical image.

13. The method of claim 9, wherein in response to the analyzing the cause to be a moving speed of the probe being above a level, the generating comprises generating the guide information of the moving speed of the probe.

14. The method of claim 9, wherein in response to the analyzing the cause to be a quality of the medical image, the generating comprises generating the guide information of at least one among an angle and a pressure of the probe.

15. The method of claim 9, further comprising outputting the generated guide information, using at least one among an acoustic method, a visual method, and a tactile method.

16. The method of claim 15, wherein the outputting comprises outputting the generated guide information as at least one among an image and a text of the operation of the probe.

17. A non-transitory computer-readable storage medium storing a program comprising instructions for causing a computer to perform the method of claim 9.

18. A computer-aided diagnosis apparatus comprising:
an image acquirer configured to acquire a medical image, using a probe;
a lesion determiner configured to determine a lesion based on the acquired medical image; and
a diagnosis supporting portion configured to:
analyze a cause of a reliability of the determined lesion being less than a level, to be at least one among a moving speed, an angle, and a pressure of the probe; and
generate guide information of at least one among the moving speed, the angle, and the pressure of the probe based on the analyzed cause.

19. The apparatus of claim 18, wherein the diagnosis supporting portion is further configured to output the generated guide information, using at least one among an acoustic method, a visual method, and a tactile method.

20. The apparatus of claim 18, wherein the diagnosis supporting portion is configured to:
analyze the cause to be the moving speed of the probe in response to the moving speed being greater than a level;
analyze the cause to be the angle of the probe in response to the angle being different than a right angle to the lesion; and
analyze the cause to be the pressure of the probe in response to the pressure being less than a level.
